(19) United States
(12) Patent Application Publication
(10) Pub. No.: US 2009/0216171 A1
(43) Pub. Date: Aug. 27, 2009

(54) Scrotal support garment

(76) Inventor: Donald R. Quinn, Bristol, TN (US)

Correspondence Address: LITMAN LAW OFFICES, LTD., POST OFFICE BOX 15035, CRYSTAL CITY STATION, ARLINGTON, VA, 22215-0035, US

(21) Appl. No.: 12/071,878

(22) Filed: Feb. 27, 2008

(51) Int. Cl. A61F 13/00; A41D 13/00; A41B 9/02; A61F 13/15

(52) U.S. Cl. 602/70; 2/466; 2/403; 604/358

(57) ABSTRACT

The scrotal support garment includes a fabric material that forms a cup to support a wearer's scrotal sack and contents thereof. The cup has a wide upper end that tapers to a narrow lower end. An elongated thong extends from the lower end of the cup. In one embodiment, the thong bifurcates to form left and right shoulder straps that are unitary members extending to opposite sides of the upper end of the cup. In another embodiment, the thong bifurcates to form a first portion of left and right shoulder straps. Elongated second portions of the left and right shoulder straps extend from opposite sides of the cup. The first and second portions are joined by adjustment straps or releasable fasteners, such as buckles, so that the length of the shoulder straps is adjustable.

### BACKGROUND OF THE INVENTION

1. Field of the Invention

The present invention relates to medical appliances and support devices for male genitalia, and particularly to a scrotal support garment that provides support for the scrotum for patients having ailments or medical conditions affecting the genitalia or groin in order to relieve pain or discomfort.

2. Description of the Related Art

Diseases such as penile cancer, testicular cancer, or the like and medical conditions resulting from a vasectomy, penectomy, or similar surgery can cause the male scrotum to swell or enlarge to an abnormal size. An enlarged or swollen scrotum is usually very painful and can render such simple activities as walking unbearable. Moreover, the edematous scrotum may distress surrounding tissues and structures, which may lead to further damage, such as tearing of the skin or scrotal sac. Although there are many devices for supporting the male genitalia, none are entirely satisfactory for relieving the pain and discomfort resulting from medical conditions affecting the genitalia or groin.

Thus, a scrotal support garment solving the aforementioned problems is desired.

### SUMMARY OF THE INVENTION

The scrotal support garment includes a fabric material that forms a cup to support a wearer's scrotal sack and contents thereof. The cup has a wide upper end that tapers to a narrow lower end. An elongated thong extends from the lower end of the cup. In one embodiment, the thong bifurcates to form left and right shoulder straps that are unitary members extending to opposite sides of the upper end of the cup. In another embodiment, the thong bifurcates to form a first portion of left and right shoulder straps. Elongated second portions of the left and right shoulder straps extend from opposite sides of the cup. The first and second portions are joined by adjustment straps or releasable fasteners, such as buckles, so that the length of the shoulder straps is adjustable.

These and other features of the present invention will become readily apparent upon further review of the following specification and drawings.

### BRIEF DESCRIPTION OF THE DRAWINGS

FIG. 1 is an environmental, perspective view of a scrotal support garment according to the present invention as viewed from the front.

FIG. 2 is an environmental, perspective view of the scrotal support garment according to the present invention as viewed from the rear.

FIG. 3 is a perspective view of the scrotal support garment according to the present invention.

Similar reference characters denote corresponding features consistently throughout the attached drawings.

### DETAILED DESCRIPTION OF THE PREFERRED EMBODIMENT

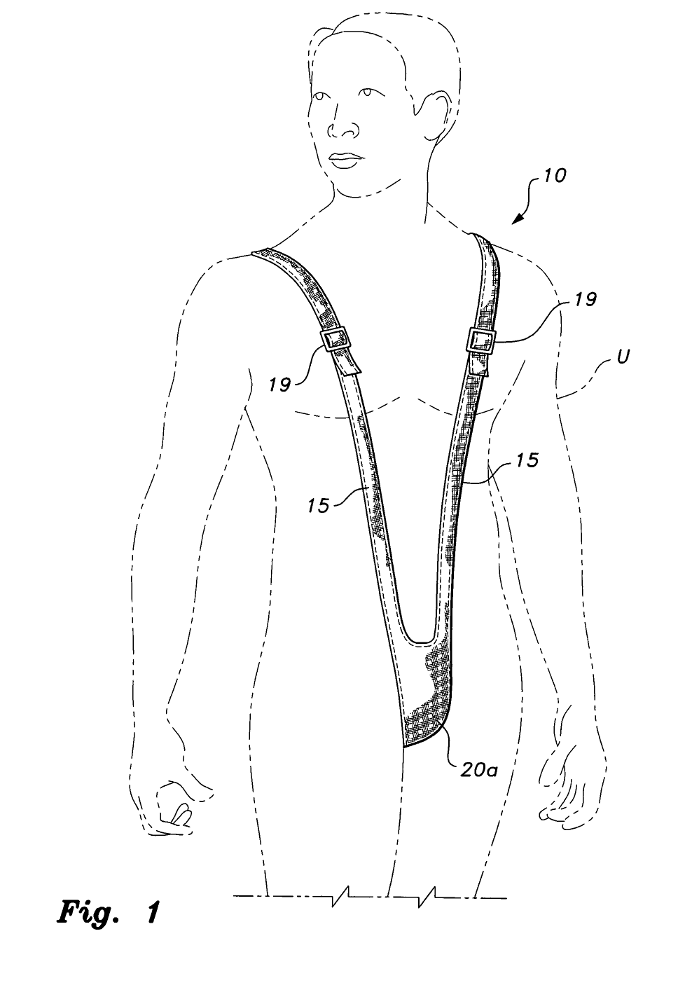

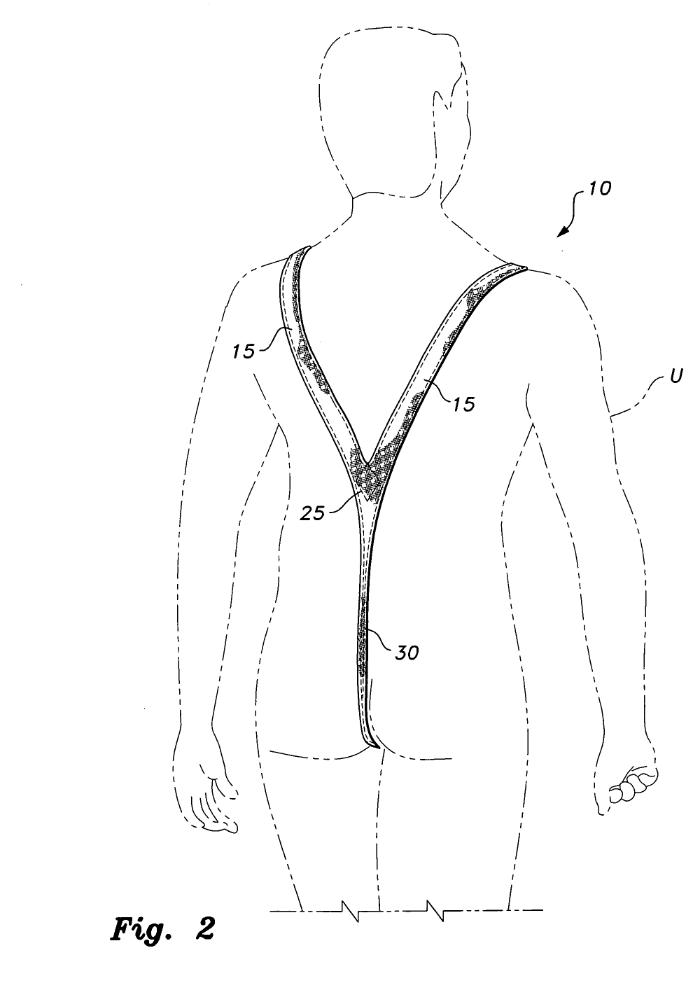

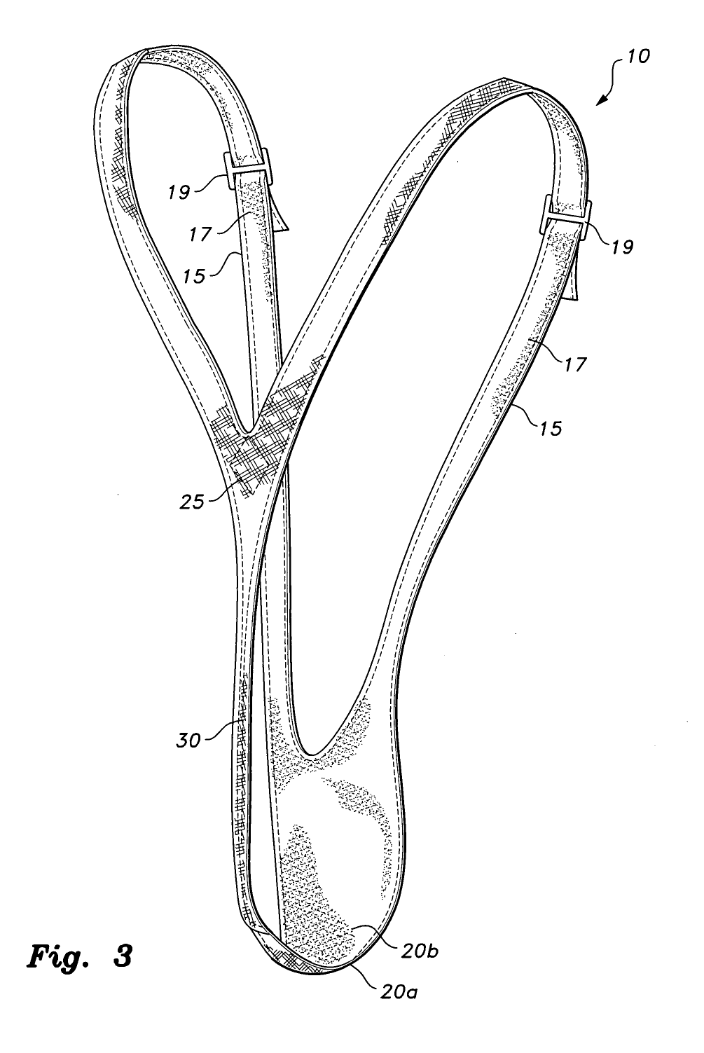

As shown in FIGS. 1-3, the present invention relates to a scrotal support garment 10 made from a flexible, preferably resilient material that forms a cup 20a, having an inside portion 20b that holds the scrotal sack of wearer U to support the scrotum. The cup inside portion 20b may have a liner made of an air permeable, liquid absorbent material to promote comfort and hygiene of the wearer. The cup material may be natural cloth, e.g., cotton, or it may be synthetic material, or a blend of cloth and synthetic material. The cup 20a has a wide upper end, the cup tapering to form a narrow lower end.

An elongate, single thong 30 extends from the lower end of the cup 20a. In one embodiment, the thong 30 bifurcates to form unitary left and right shoulder straps 15 that are attached to opposite sides of the upper end of the cup 20a. The shoulder straps 15 may be made from elastic material. In another embodiment, the thong 30 is elongated to form a single strap to extend up the user's back U′ and bifurcates to form first portions of left and right shoulder straps 15. Second portions of the left and right shoulder straps 15 extend from opposite sides of the cup's upper end. The first and second portions of the left and right shoulder straps 15 are joined by an adjustment strap or releasable fastener 19, such as a buckle or hook and loop fasteners, that permits adjustment of the length of the straps 15. In both embodiments, a diamond-shaped reinforcement 25 is stitched to the junction at which the shoulder straps 15 bifurcate from the thong 30. The shoulder straps 15 may be lined with elastic strips 17 or bands.

It is to be understood that the present invention is not limited to the embodiment described above, but encompasses any and all embodiments within the scope of the following claims.

I claim:

1. A scrotal support garment, comprising:
a flexible cup adapted for supporting a scrotum, the cup having a wide upper end, the cup tapering to form a narrow lower end;
an elongated thong extending from the lower end of the cup; and
left and right shoulder straps extending between the thong and opposite sides of the upper end of the cup.

2. The scrotal support garment according to claim 1, wherein the cup is made of a fabric material.

3. The scrotal support garment according to claim 1, wherein the shoulder straps are unitary members.

4. The scrotal support garment according to claim 3, wherein the shoulder straps are made from an elastic material.

5. The scrotal support garment according to claim 1, wherein said shoulder straps comprise a first portion extending from said thong and a second portion extending from the upper end of said cup, the scrotal support garment further comprising adjustment straps joining the first and second portions of said shoulder straps.

6. The scrotal support garment according to claim 1, wherein said shoulder straps comprise a first portion extending from said thong and a second portion extending from the upper end of said cup, the scrotal support garment further comprising releasable fasteners joining the first and second portions of said shoulder straps.

7. The scrotal support garment according to claim 1, wherein said thong bifurcates at a junction to form said left and right shoulder straps, the scrotal support garment further comprising a diamond-shaped reinforcement patch attached to the junction.

8. The scrotal support garment according to claim 1, further comprising a liner disposed in said cup, the liner being made from an air permeable, liquid absorbent material.

9. A scrotal support garment, comprising:
a flexible cup adapted for supporting a scrotum, the cup having a wide upper end, the cup tapering to form a narrow lower end;
an elongated thong extending from the lower end of the cup;
left and right shoulder straps extending between the thong and opposite sides of the upper end of the cup; and
means for adjusting the length of the left and right shoulder straps.

10. The scrotal support garment according to claim 9, wherein said left and right shoulder straps comprise a first portion extending from said thong and a second portion extending from said cup, said length adjusting means comprising an adjustable strap disposed between the first and second portions.

11. The scrotal support garment according to claim 9, wherein said left and right shoulder straps comprise a first portion extending from said thong and a second portion extending from said cup, said length adjusting means comprising a releasable fastener disposed between the first and second portions.
